(19) United States
(12) Patent Application Publication
(10) Pub. No.: US 2012/0202286 A1
(43) Pub. Date: Aug. 9, 2012

(54) rbLIF PROTEIN FOR USE IN EMBRYONIC STEM CELL CULTURES

(76) Inventors: Jie Xu, Centreville, VA (US); Jerry Yang, (US); Xiuchun Tian, Storrs, CT (US); Fuliang Du, Mansfield, CT (US); Yinghong Ma, Middletown, CT (US)

(21) Appl. No.: 13/451,268

(22) Filed: Apr. 19, 2012

Related U.S. Application Data
(62) Division of application No. 12/637,660, filed on Dec. 14, 2009.
(60) Provisional application No. 61/122,670, filed on Dec. 15, 2008.

(51) Int. Cl. C12N 5/0735; C12N 5/02

(52) U.S. Cl. 435/354; 435/377; 435/366

(57) ABSTRACT

Provided herein methods of maintaining an embryonic stem cell culture in an undifferentiated state, wherein the method includes culturing an embryonic stem cell on a rabbit feeder cell layer, wherein the rabbit feeder layer expresses a leukemia inhibitory factor (LIF) protein. Also provided are methods of maintaining an embryonic stem cell culture in an undifferentiated state, wherein the method includes culturing an embryonic stem cell in a culture medium comprising rabbit leukemia inhibitory factor (rbLIF) protein. Also provided are polynucleotide and amino acid sequences of a rabbit leukemia inhibitory factor protein (rbLIF).

### RELATED APPLICATION DATA

This application is a divisional application of U.S. application Ser. No. 12/637,660, filed Dec. 14, 2009, which claims the benefit of priority under 35 U.S.C. §119(e) of U.S. Ser. No. 61/122,670, filed Dec. 15, 2008, the entire contents of each is incorporated herein by reference.

### FIELD OF THE INVENTION

The present invention relates generally to culturing of embryonic stem cells, and more specifically to the use of rbLIF protein in cell culture.

### BACKGROUND OF THE INVENTION

Embryonic stem cells (“ES cells” or ESCs) are derived from the inner cell mass (ICM) of fertilized eggs in blastocyst phase, and can be cultured and maintained in vitro while being kept in an undifferentiated state. ES cells are pluripotent, possessing the capability of developing into any organ or tissue type or, at least potentially, into a complete embryo. For example, ES cells can differentiate and give rise to a succession of mature functional cells. Differentiation has been shown in tissue culture and in vivo.

ES cells have been derived from various species including mouse, human, bovine, and rabbit. However, the culture conditions ES cells from different species can vary, for example, culture conditions for human ES cells differ from the culture conditions for the mouse ES cell. Moreover, rabbit ES cells have proven to be more difficult to culture than ES cells from other species.

Thus, there is a need in the art for improved methods for maintaining rabbit ES cells.

### SUMMARY OF THE INVENTION

The present invention is based on the discovery that rabbit leukemia inhibitory factor (rbLIF) has surprisingly beneficial effects when used in culturing embryonic stem cells, particularly rabbit embryonic stem cells. Accordingly, provided herein are methods of maintaining an embryonic stem cell culture in an undifferentiated state, wherein the method includes culturing an embryonic stem cell on a rabbit feeder cell layer, wherein the rabbit feeder cell layer expresses a leukemia inhibitory factor (LIF) protein. In some embodiments, the rabbit feeder cell layer is isolated from embryonic tissues from an embryo obtained from a transgenic rabbit. In preferred embodiments, the transgenic rabbit expresses, or overexpresses, a selection marker, such as a neomycin resistance gene.

In another embodiment of the present invention, there are provided methods of maintaining an embryonic stem cell culture in an undifferentiated state, wherein the method includes culturing an embryonic stem cell in a culture medium comprising rabbit leukemia inhibitory factor (rbLIF) protein.

In still another embodiment of the present invention, there is provided an isolated rabbit leukemia inhibitory factor protein (rbLIF). In another embodiment there is provided a polynucleotide encoding rbLIF.

Illustrative embodiments of the invention are further described in Attachment 1, the entire content of which are incorporated herein by reference.

### BRIEF DESCRIPTION OF THE DRAWINGS

FIG. 1 is a comparison of the coding sequences from LIF cDNA from humans, mice and rabbits (SEQ. ID. Nos 1, 2 and 3, respectively).

FIG. 2A is the complete rbLIF cDNA sequence from rabbits (SEQ. ID. No. 4, with coding regions underlined), followed by FIG. 2B, the decoded open reading frame for rbLIF (SEQ. ID. No. 5).

FIG. 3 depicts gels demonstrating the presence of the rbLIF sequence of SEQ. ID. No. 5 (obtained through BLAST) confirmed as present in three individual rabbits.

FIG. 4A shows the polypeptide sequence of rbLIF (SEQ. ID. No. 5) in comparison to the LIF protein sequence of humans, mice and cattle (SEQ. ID. Nos. 6, 7 and 8, respectively). As shown in the homology table of FIG. 4B, the sequence in the rabbit is closer to that of humans than mice or cattle.

FIG. 5 is a plasmid map for the synthesis of rbLIF (SEQ. ID. Nos. 9 and 10).

FIG. 6 depicts a gel with digested pGEX-rbLIF (1% agarose).

FIG. 7A is a SDS PAGE gel staining for GST fusion protein, while FIG. 7B depicts a Western blot for anti-hLIF binding.

FIG. 8 shows mouse embryonic stem cells (mESC) cultured on mouse feeder cells in the presence of different concentrations of rbLIF (20 ng/ml, 4 ng/ml, 2 ng/ml and 0 ng/ml, in panels I, II, III and IV, respectively.

FIG. 9 shows that rbESC colonies were established (here, on rabbit feeder cells supplemented with 20 ng/ml rbLIF, panel I) to a greater extent in the presence of rbLIF than in the absence of rbLIF (panel II).

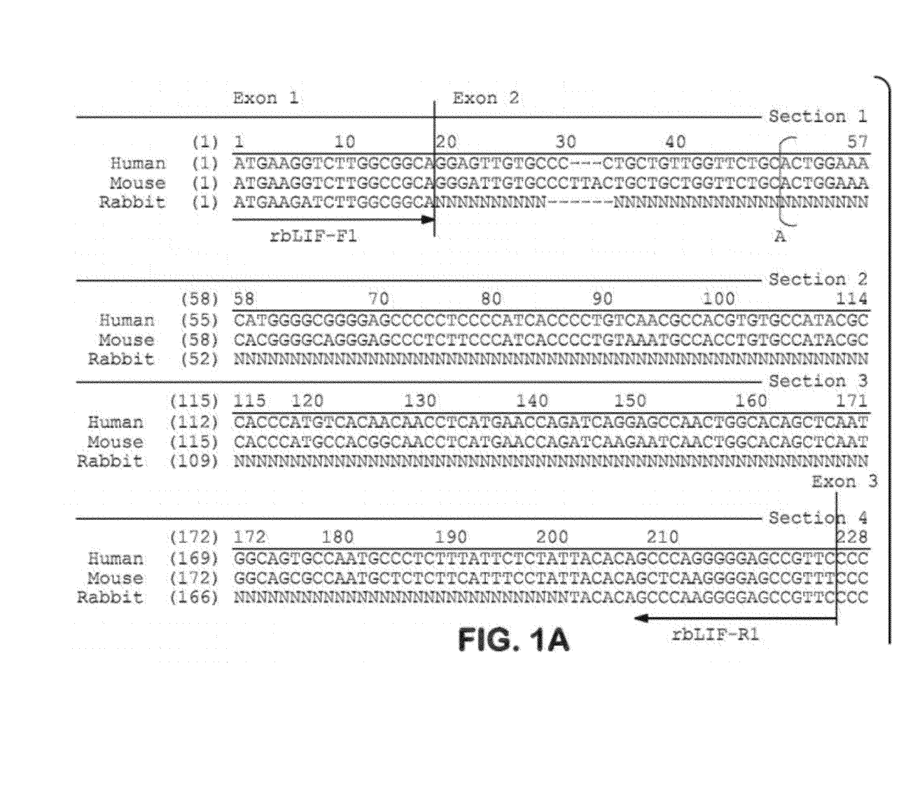

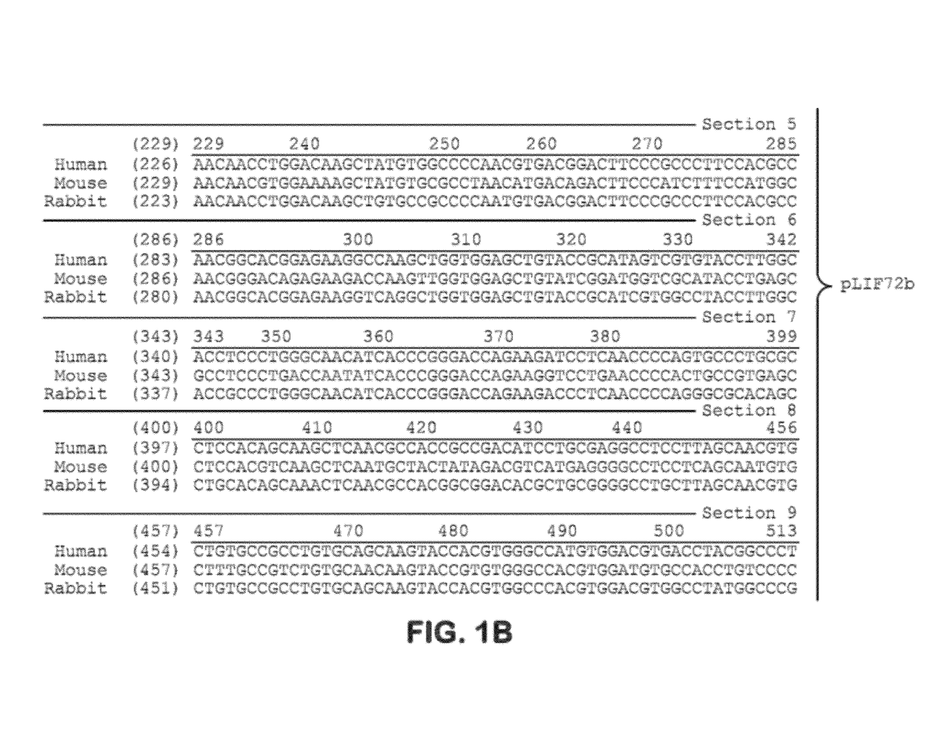

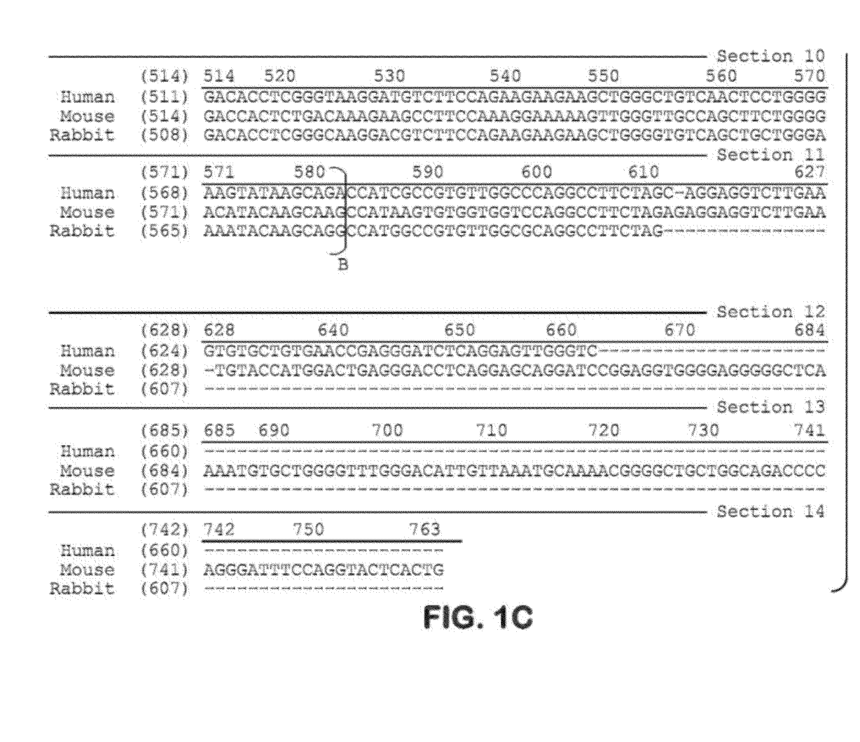

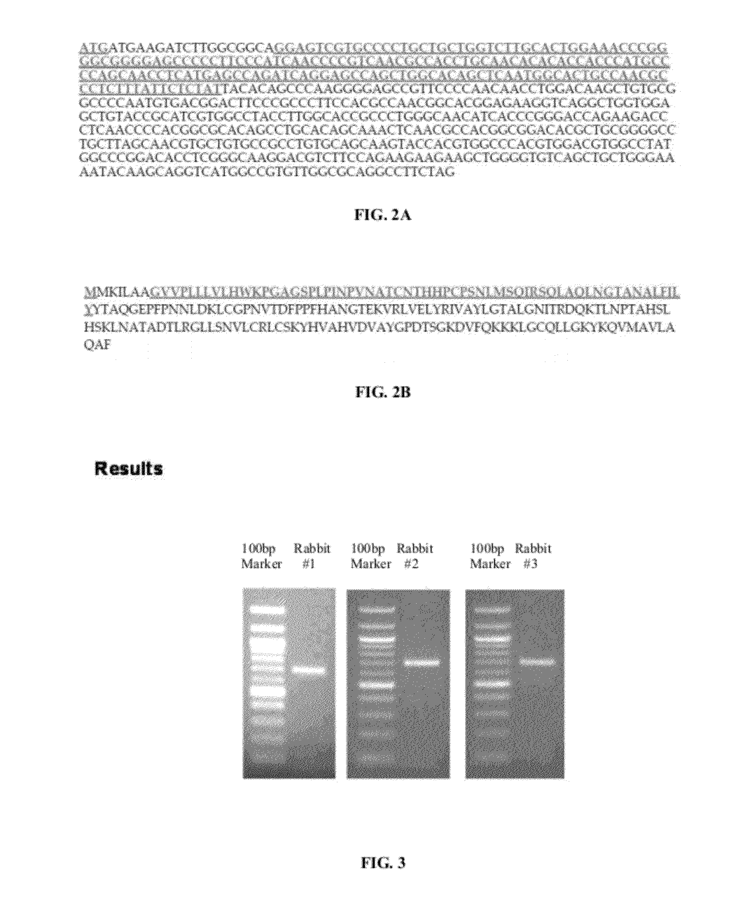

### DETAILED DESCRIPTION OF THE INVENTION

Before the present methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. References cited in this disclosure are hereby incorporated by this reference within it.

The present invention is based on the discovery that rabbit leukemia inhibitory factor (rbLIF) has surprisingly beneficial effects when used in culturing embryonic stem cells, particularly rabbit embryonic stem cells, which are somewhat more difficult to maintain than the embryonic stem cells of other mammals. Without wishing to be bound by any particular theory, it is believed that the presence of rbLIF improves the robustness of the culture to an unexpected degree and allows improved germline development.

In accordance with the present invention, there are provided rabbit leukemia inhibitory factor (rbLIF) proteins having the following sequence:

(SEQ ID NO: 5)
MMKILAAGVVPLLLVLHWKPGAGSPLPINPVNATCNTHHPCPSNLM
SQIRSQLAQLNGTANALFILYYTAQGEPFPNNLDKLCGPNVTDFPP
FHANGTEKVRLVELYRIVAYLGTALGNITRDQKTLNPTAHSLHSKL
NATADTLRGLLSNVLCRLCSKYHVAHVDVAYGPDTSGKDVFQKKKL
GCQLLGKYKQVMAVLAQAF.

In another embodiment of the invention, there are provided polynucleotides encoding rbLIF protein. In one aspect, the polynucleotide has the following sequence:

(SEQ ID NO: 4)
ATGATGAAGATCTTGGCGGCAGGAGTCGTGCCCCTGCTGCTGGTCTT
GCACTGGAAACCCGGGGCGGGGAGCCCCCTTCCCATCAACCCCGTCA
ACGCCACCTGCAACACACACCACCCATGCCCCAGCAACCTCATGAGC
CAGATCAGGAGCCAGCTGGCACAGCTCAATGGCACTGCCAACGCCCT
CTTTATTCTCTATTACACAGCCCAAGGGGAGCCGTTCCCCAACAACC
TGGACAAGCTGTGCGGCCCCAATGTGACGGACTTCCCGCCCTTCCAC
GCCAACGGCACGGAGAAGGTCAGGCTGGTGGAGCTGTACCGCATCGT
GGCCTACCTTGGCACCGCCCTGGGCAACATCACCCGGGACCAGAAGA
CCCTCAACCCCACGGCGCACAGCCTGCACAGCAAACTCAACGCCACG
GCGGACACGCTGCGGGGCCTGCTTAGCAACGTGCTGTGCCGCCTGTG
CAGCAAGTACCACGTGGCCCACGTGGACGTGGCCTATGGCCCGGACA
CCTCGGGCAAGGACGTCTTCCAGAAGAAGAAGCTGGGGTGTCAGCTG
CTGGGAAAATACAAGCAGGTCATGGCCGTGTTGGCGCAGGCCTTCTA
G.

In still another embodiment of the invention, there are provided methods of maintaining an embryonic stem cell culture in an undifferentiated state, wherein the method includes culturing an embryonic stem cell on a rabbit feeder cell layer, wherein the rabbit feeder layer expresses a leukemia inhibitory factor (LIF) protein. In some embodiments, the embryonic stem cells are from rabbit. In other embodiments, the embryonic stem cells are from mouse.

As used herein, the terms “maintain” or “maintenance” refer to the stable preservation of the characteristics of the stem cells when cultured in specific culture conditions. Such characteristics can include the cell morphology and gene expression profiles of the stem cells, which can be determined using the techniques described herein. The term “maintain” can also encompass the propagation of the cells, or an increase in the number of ES cells being cultured. The invention contemplates culture conditions that permit the ES cells to remain pluripotent, while the ES cells may or may not continue to divide and increase in number.

The term “feeder cell” refers to a culture of cells that grows in vitro and can be used to support the growth of another cell of interest in culture, such as an embryonic stem cell. As used herein, a “feeder cell layer” can be used interchangeably with the term “feeder cell.” A feeder cell can comprise a monolayer, where the feeder cells cover the surface of the culture dish with a complete layer before growing on top of each other, or can comprise clusters of cells. As used herein, the terms “cluster” and “clump” can be used interchangeably, and generally refer to a group of cells that have not been dissociated into single cells. In some embodiments, the feeder cell comprises an adherent monolayer. Additionally, the cell of interest may or may not be cultured in direct contact with the feeder cell. For instance, the cell of interest can be co-cultured with the feeder cell in such a manner that the cell of interest is physically separated from the feeder cell by a membrane containing pores, yet the feeder cell still enriches the medium in such a way as to support the growth of the cell of interest.

Rabbit feeder layers for use in the invention methods may be any cell type typically used as a feeder cell from rabbit, provided rbLIF is expressed. In certain embodiments, the rabbit feeder layer is prepared from rabbit embryonic tissues by methods known in the art. Embryonic tissues may be obtained from the embryos of wild type rabbits or from transgenic rabbits. In one aspect, the embryonic tissues are obtained from the embryos of a transgenic rabbit expressing a selection marker such as an antibiotic resistance gene. In another aspect, the embryonic tissues are obtained from a transgenic rabbit expressing a transgene encoding a LIF protein and optionally a selection marker. In particular embodiments, the feeder layer is isolated from embryonic tissues of rabbit embryos of less than 3 weeks in development (e.g., as utilized in connection with the experiments whose results are shown in FIG. 9). In other embodiments, the feeder layer is isolated from embryonic tissues of rabbit embryos of less than 2 weeks in development.

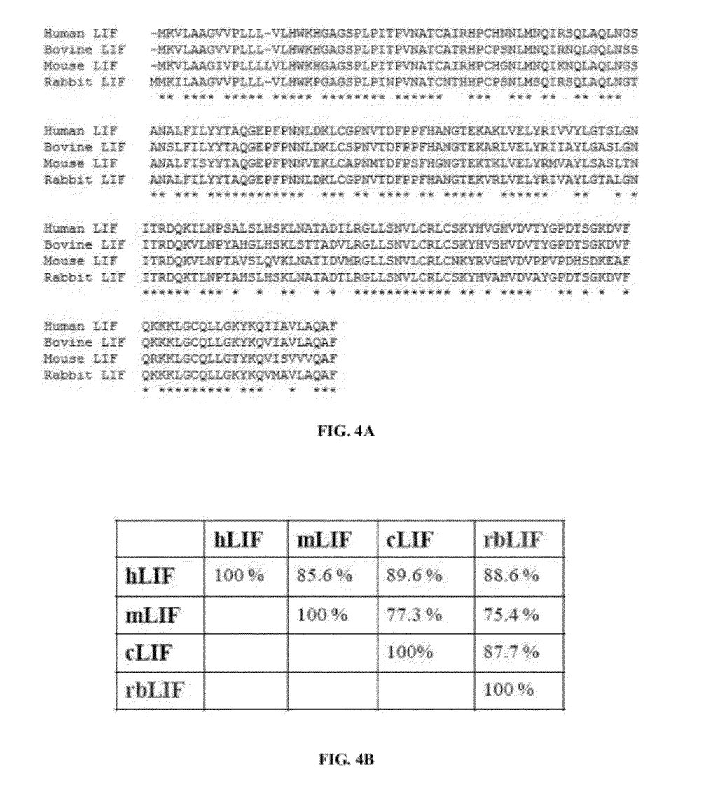

In some embodiments, the feeder layer expresses endogenous rabbit LIF. In one aspect, the feeder layer expresses endogenous rabbit LIF and a transgene encoding a selection marker. In other embodiments, the feeder layer expresses LIF protein encoded by a transgene. The transgene may encode rbLIF, or may encode LIF from another species. For example, the amino acid sequences of LIF proteins from other species (e.g., human, mouse, and bovine) have been reported in the literature (see, e.g., the human, mouse and bovine sequences in FIG. 4, SEQ. ID. Nos: 6, 7 and 8). In certain embodiments, rabbit ES cells are grown on rabbit feeder cells expressing a transgene encoding rbLIF. In further embodiments, ES cells from other species may be grown on rabbit feeder cells expressing a transgene encoding LIF from the same species as the ES cells. For example, human ES cells may be grown on rabbit feeder cells expressing human LIF.

Accordingly, there are provided herein transgenic rabbit expressing a transgene. In some embodiments the transgene encodes an LIF protein. In other embodiments, the transgene encodes a selection marker, such as an antibiotic resistance gene. In particular embodiments, the genome of the transgenic rabbit contains an expression cassette including a transgene encoding a leukemia inhibitory factor (LIF) protein. In certain embodiments, the transgene encodes an LIF protein from a species selected from the group consisting of rabbit, human, mouse, and rat. In one aspect, the transgene encodes rbLIF and the transgenic rabbit overexpresses rbLIF. In some embodiments, the expression cassette further includes a selection marker.

Also provided are progeny of a transgenic rabbit of the invention. In one aspect, the progeny genome contains the transgene encoding the LIF protein. In another aspect, the progeny genome contains a transgene encoding a selection marker.

Further provided are embryos obtained from the transgenic rabbits of the invention. In one aspect, the embryo genome contains the transgene encoding the LIF protein. In another aspect, the embryo genome contains a transgene encoding a selection marker. In other embodiments there are provided cells isolated from such embryos, wherein the embryo is less than 3 weeks of development, wherein the cell expresses LIF protein. In particular embodiments, the embryonic cells further express a selection marker.

Transgenic rabbits expressing a transgene can be generated by methods known in the art (e.g., U.S. Pat. No. 6,512,161; and Duverger, et al., Arterioscler Thromb Vasc Biol (1996) 16, 1424-1429). For example, a transgenic rabbit may be generated by injecting, into a rabbit embryo, exogenous DNA sequences encoding LIF protein, particularly rbLIF, transferring the embryo to a recipient rabbit and, after the birth, checking for the presence of the DNA sequences in the genome of the neonate rabbits.

The methods enabling the introduction of DNA into cells are generally available and well-known in the art. Different methods of introducing transgenes could be used. Generally, the zygote is the preferred target for microinjection. In most cases, the injected DNA will be incorporated into the host gene before the first cleavage (Brinster, et al., (1985) Proc. Natl. Acad. Sci. USA 82, 4438-4442). Consequently, nearly all cells of the transgenic non-human animal will carry the incorporated transgene. Generally, this will also result in the efficient transmission of the transgene to offspring of the founder since 50% of the germ cells will harbor the transgene.

Retroviral infection can also be used to introduce a transgene. The developing rabbit embryo can be cultured in vitro to the blastocyst stage. During this time, blastomeres may be targets for retroviral infection (Jaenich, R. (1976) Proc. Natl. Acad. Sci. USA 73, 1260-1264). Efficient infection of the blastomeres is obtained by enzymatic treatment to remove the zona pellucida (Hogan et al., (1986) in Manipulating the Mouse Embryo, Cold Spring Harbor Laboratory Press, Cold Spring Harbor, N.Y.). The viral vector system used to introduce the transgene is typically a replication-defective retrovirus carrying the transgene (Jahner et al., (1985) Proc. Natl. Acad. Sci. USA 82, 6927-6931; Van der Putten et al., (1985) Proc. Natl. Acad. Sci. USA 82, 6148-6152). Transfection is easily and efficiently obtained by culturing the blastomeres on a monolayer of virus-producing cells (Van der Putten et al., (1985) Proc. Natl. Acad. Sci. USA 82, 6148-6152; Stewart et al., (1987) EMBO J. 6:383-388). Alternatively, infection can be performed at a later stage. Virus or virus-producing cells can be injected into the blastocoele (Jahner et al., (1982) Nature 298:623-628.). Most of the founder animals will be mosaic for the transgene since incorporation occurs only in a subset of the cells which formed the transgenic non-human animal. Furthermore, the founder animal may contain retroviral insertions of the transgene at a variety of positions in the genome; these generally segregate in the offspring. In addition, it is also possible to introduce transgenes into the germ line, albeit with low efficiency, by intrauterine retroviral infection of the midgestation embryo (Jahner et al., (1982) Nature 298:623-628.).

A third type of target cell for transgene introduction is the embryonal stem cell (ES). ES cells are obtained from pre-implantation embryos cultured in vitro (Evans, M. J., et al., (1981) Nature 292, 154-156; Bradley, A., et al. (1984) Nature 309, 255-258; Gossler, et al., (1986) Proc. Natl. Acad. Sci. USA 83, 9065-9060; and Robertson, et al., (1986) Nature 322, 445-448). Transgenes can be efficiently introduced into ES cells by DNA transfection or by retrovirus-mediated transduction. The resulting transformed ES cells can thereafter be combined with blastocysts from a non-human animal. The ES cells colonize the embryo and contribute to the germ line of the resulting chimeric animal (For review see Jaenisch, R. (1988) Science 240, 1468-1474).

To be expressed, a transgene should be operably linked to a regulatory region. Regulatory regions, such as promoters, may be used to increase, decrease, regulate or designate to certain tissues or to certain stages of development the expression of a gene. Selection of the appropriate regulatory region or regions is a routine matter that is within the level of ordinary skill in the art.

The regulatory regions may comprise a promoter region for functional transcription, as well as a region situated in 3′ of the gene of interest, and which specifies a signal for termination of transcription and a polyadenylation site. All these elements constitute an expression cassette.

Promoters that may be used in the present invention include both constitutively active promoters and inducible promoters. The promoter may be naturally responsible for the expression of the nucleic acid, or may be from a heterologous source. Promoter sequences may be from eukaryotic or viral genes. In certain embodiments, the promoter sequence is derived from the genome of the cell in which it is desired to infect. Alternatively, the promoter sequence may be derived from the genome of a virus. In this regard, the promoters of the E1A, MLP, HCMV and RSV genes may be used. In addition, the promoter may be modified by addition of activating or regulatory sequences or sequences allowing a tissue-specific or predominant expression.

Additional promoters useful for practice of this invention are the ubiquitous promoters HPRT vimentin, actin and tubulin the intermediate filament promoters desmin neurofilaments, keratin, and GFAP; the therapeutic gene promoters MDR, CFTR, and factor VIII promoters which are preferentially activated in dividing cells; promoters which respond to a stimulus such as steroid hormone receptor and retinoic acid receptor promoters; tetracycline-regulated transcriptional modulators; cytomegalovirus immediate-early; retroviral LTR metallothionein; SV40, E1a and MLP promoters. Tetracycline-regulated transcriptional modulators and CMV promoters are described in WO 96/01313, U.S. Pat. Nos. 5,168,062 and 5,385,839, the contents of which are incorporated herein by reference.

Expression cassettes may further contain one or more selection markers for ease of isolation and culturing of cells expressing the transgene encoding the LIF protein. Such markers are well known in the art, and include for example, antibiotic resistance genes. In one example, the expression cassette includes a neomycin resistance gene. Such transgenic rabbits may be used for generation of embryos from which feeder cells are isolated. The presence of a neomycin resistance marker facilitates the isolation and culture of feeder cells which express rbLIF. In another embodiment, the selection marker is a fluorescent protein.

Methods for evaluating the presence of the introduced DNA as well as its expression are readily available and well-known in the art. Such methods include, but are not limited to DNA (Southern) hybridization to detect the exogenous DNA, polymerase chain reaction (PCR), polyacrylamide gel electrophoresis (PAGE) and Western blots to detect DNA, RNA and protein.

Embryonic stem cells may be isolated by methods known in the art, including the isolation of rabbit embryonic stem cells (e.g., U.S. Pat. No. 6,103,523). In one example, superovulated Dutch Belted females are mated with Dutch Belted males. The blastocysts are isolated on day 5.5 and rinsed with Dulbecco's phosphate buffered saline supplemented with 3% bovine serum albumin (Cohn fraction V) plus 5% (v/v) antibiotic/antimycotic solution. The blastocysts are incubated in rabbit ES medium at 39° C., 5% CO₂. The mucin coat and zona pellucida of the blastocysts are removed using acidified phosphate buffered saline (pH 2.5) and 0.5% pronase in phosphate buffered saline. The inner cell mass is prepared manually out of the surrounding trophectoderm cells with 2 needles and placed individually in a 96 well culture dish (plated with rabbit feeder cells with a density equivalent to 3 to 4×10 cells per 10 cm dish).

The explanted inner cell masses are refed daily with rabbit ES cell medium. After 2 days, the inner cell mass outgrowth can be freed from remaining trophectoderm cells by gently lifting the trophectoderm outgrowth with a beveled glass pipet off the underlying feeder layer and by aspirating it into the glass pipet. ES-like colonies, characterised by three dimensional growth are subsequently passaged onto new culture dishes. The culture plate is selectively trypsinized after 4 to 5 days, thus allowing the selective passaging of ES cells and not of trophectoderm cells or feeder cells. The ES cells are passaged very gradually onto larger culture dishes at 4 to 5 days intervals, to maintain the ES cells at a very high density, another prerequisite to prevent differentiation and loss of pluripotency. The feeder densities on the subsequent culture dishes are maintained at densities equivalent of 3 to 4×10 cells per 10 cm dish.

In another embodiment of the present invention, there are provided methods of maintaining an embryonic stem cell culture in an undifferentiated state, wherein the method includes culturing an embryonic stem cell in a culture medium (using rabbit or non-rabbit feeder cells) comprising rabbit leukemia inhibitory factor (rbLIF) protein (e.g., 20 ng/ml., 4 ng/ml or 2 ng/ml, as shown in FIG. 8). Such cells are better established in the presence of rbLIF than are cells in the absence of rbLIF (see, FIG. 8, panel IV and FIG. 9, panel II).

In some embodiments, the medium for culture of rabbit embryonic stem cells comprises high glucose Dulbecco's Modified Eagle Medium, 4 mM L-glutamine, 0.1 mM 2-mercaptoethanol, 148 units/ml penicillin G sodium, 148 microgram/ml streptomycin sulfate, 4 microgram/ml bovine insulin, 20% fetal bovine serum, 1.5% MEM non-essential amino acid solution. Culture medium may be supplemented with LIF protein, when ES cells are grown in the absence of a rabbit feeder cell layer secreting rbLIF. In one aspect, the culture medium is supplemented with rbLIF. In other embodiments, culture medium may be further supplemented with rbLIF when ES cells are grown in the presence of a rabbit feeder layer.

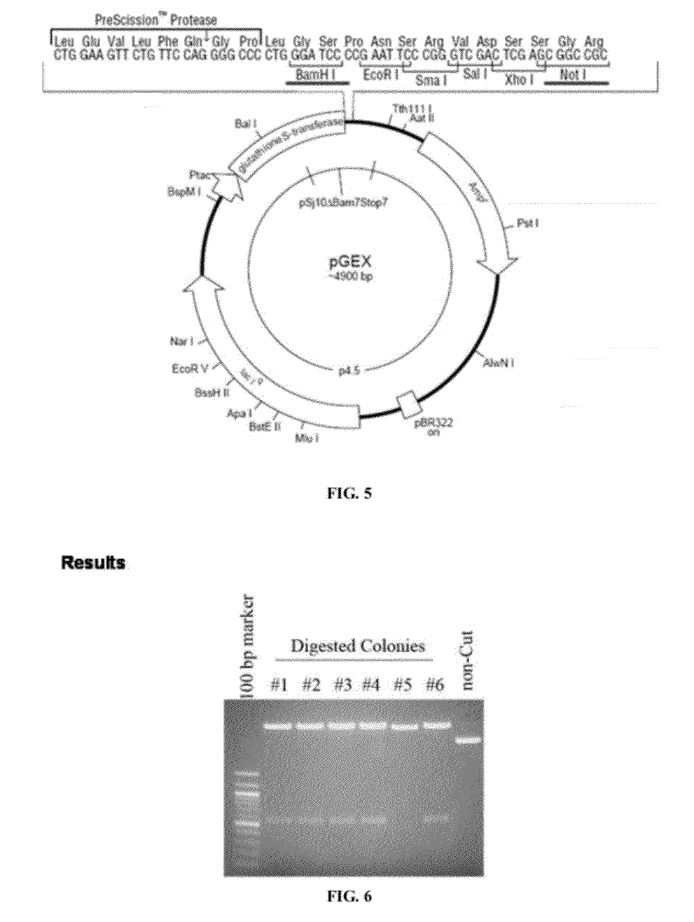

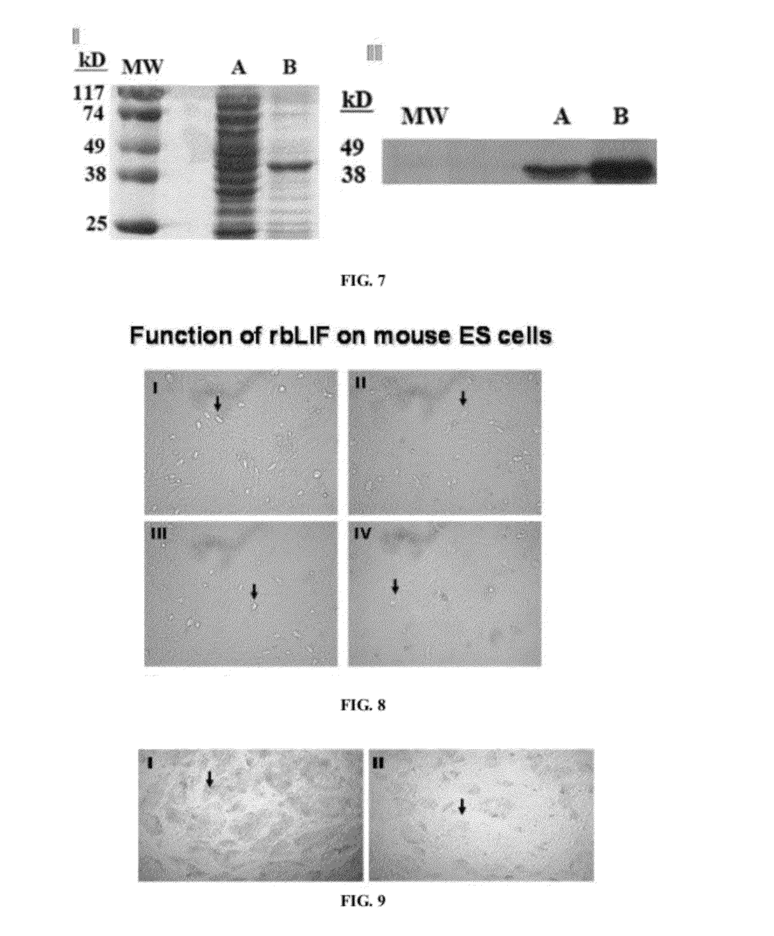

In some embodiments, rbLIF may be produced by recombinant methods known in the art. For example, recombinant rbLIF may be produced in E. coli systems. Such rbLIF so produced may be isolated and purified and used to supplement culture media. For example, with the complete cDNA sequence of rbLIF (SEQ. ID.No:3), an E. coli system was used to synthesize recombinant rbLIF. To that end, a rbLIF frangment was cloned into a pGEX 6P-1 vector by insertion with BamH1 and Not1 into a plasmid (see, FIG. 5). Expression of a GEX-rbLIF fusion protein (GST) was monitored and optimized. Recombinant cells (500 L) were cultured, harvested and lysed. The GST fusion protein was purified. As shown in FIG. 6, 5 colonies (1, 2, 3, 4 and 6) were sequenced to confirm the presence of rbLIF in the colony, and colony 3 was chosen for use in expressing the recombinant protein. The expression was confirmed using an anti-rbLIF antibody, as shown in FIG. 7.

In certain embodiments, ES cells may be grown in conditioned medium containing rbLIF protein. As used herein “conditioned medium” refers to a cell culture medium that is obtained from a culture of a rabbit feeder cell layer on which ES cells can be cultured and maintained in a pluripotent state. Culture medium compositions typically include essential amino acids, salts, vitamins, minerals, trace metals, sugars, lipids and nucleosides. Cell culture medium attempts to supply the components necessary to meet the nutritional needs required to grow cells in a controlled, artificial and in vitro environment. Nutrient formulations, pH, and osmolarity vary in accordance with parameters such as cell type, cell density, and the culture system employed. Many cell culture medium formulations are documented in the literature and a number of media are commercially available. Once the culture medium is incubated with cells, it is known to those skilled in the art as “spent” or “conditioned medium”. Conditioned medium contains many of the original components of the medium, as well as a variety of cellular metabolites and secreted proteins, including, for example, biologically active growth factors, inflammatory mediators and other extracellular proteins provided by the cultured feeder layer. In particular embodiments, the conditioned medium contains LIF protein secreted by the feeder layer. The cells used to condition the medium may be genetically modified to alter the concentration of proteins found in the medium.

Although the invention has been described above and with reference to Attachment 1, the entire content of which are incorporated herein by reference, it will be understood that modifications and variations are encompassed within the spirit and scope of the invention. Accordingly, the invention is limited only by the following claims.

We claim:

1. A method of maintaining an embryonic stem cell culture in an undifferentiated state, comprising culturing mammalian embryonic stem cell on a rabbit feeder cell layer, wherein the rabbit feeder cell layer expresses leukemia inhibitory factor (rbLIF) protein.

2. The method of claim 15, wherein the embryonic stem cells are from rabbit.

3. The method of claim 15, wherein the embryonic stem cells are from mouse.

4. The method of claim 15, wherein the feeder cell layer expresses a selection marker.

5. The method of claim 15, wherein the mammalian embryonic stem cell is a murine, rabbit, porcine or human stem cell.


SEQUENCE LISTING (10 sequences)

SEQ ID NO: 1 - DNA, 659 bp, Homo sapiens
atgaaggtcttggcggcaggagttgtgcccctgctgttggttctgcactggaaacatggggcggggagccccctccccatcacccctgtcaacgccacgtgtgccatacgccacccatgtcacaacaacctcatgaaccagatcaggagccaactggcacagctcaatggcagtgccaatgccctctttattctctattacacagcccagggggagccgttccccaacaacctggacaagctatgtggccccaacgtgacggacttcccgcccttccacgccaacggcacggagaaggccaagctggtggagctgtaccgcatagtcgtgtaccttggcacctccctgggcaacatcacccgggaccagaagatcctcaaccccagtgccctgcgcctccacagcaagctcaacgccaccgccgacatcctgcgaggcctccttagcaacgtgctgtgccgcctgtgcagcaagtaccacgtgggccatgtggacgtgacctacggccctgacacctcgggtaaggatgtcttccagaagaagaagctgggctgtcaactcctggggaagtataagcagaccatcgccgtgttggcccaggccttctagcaggaggtcttgaagtgtgctgtgaaccgagggatctcaggagttgggtc

SEQ ID NO: 2 - DNA, 762 bp, Mouse
atgaaggtcttggccgcagggattgtgcccttactgctgctggttctgcactggaaacacggggcagggagccctcttcccatcacccctgtaaatgccacctgtgccatacgccacccatgccacggcaacctcatgaaccagatcaagaatcaactggcacagctcaatggcagcgccaatgctctcttcatttcctattacacagctcaaggggagccgtttcccaacaacgtggaaaagctatgtgcgcctaacatgacagacttcccatctttccatggcaacgggacagagaagaccaagttggtggagctgtatcggatggtcgcatacctgagcgcctccctgaccaatatcacccgggaccagaaggtcctgaaccccactgccgtgagcctccacgtcaagctcaatgctactatagacgtcatgaggggcctcctcagcaatgtgctttgccgtctgtgcaacaagtaccgtgtgggccacgtggatgtgccacctgtccccgaccactctgacaaagaagccttccaaaggaaaaagttgggttgccagcttctggggacatacaagcaagccataagtgtggtggtccaggccttctagagaggaggtcttgaatgtaccatggactgagggacctcaggagcaggatccggaggtggggagggggctcaaaatgtgctggggtttgggacattgttaaatgcaaaacggggctgctggcagaccccagggatttccaggtactcactg

SEQ ID NO: 3 - DNA, 606 bp, Rabbit
  misc_feature (19)..(195): n is a, c, g, or t
atgaagatcttggcggcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntacacagcccaaggggagccgttccccaacaacctggacaagctgtgccgccccaatgtgacggacttcccgcccttccacgccaacggcacggagaaggtcaggctggtggagctgtaccgcatcgtggcctaccttggcaccgccctgggcaacatcacccgggaccagaagaccctcaaccccagggcgcacagcctgcacagcaaactcaacgccacggcggacacgctgcggggcctgcttagcaacgtgctgtgccgcctgtgcagcaagtaccacgtggcccacgtggacgtggcctatggcccggacacctcgggcaaggacgtcttccagaagaagaagctggggtgtcagctgctgggaaaatacaagcaggccatggccgtgttggcgcaggccttctag

SEQ ID NO: 4 - DNA, 612 bp, Rabbit
atgatgaagatcttggcggcaggagtcgtgcccctgctgctggtcttgcactggaaacccggggcggggagcccccttcccatcaaccccgtcaacgccacctgcaacacacaccacccatgccccagcaacctcatgagccagatcaggagccagctggcacagctcaatggcactgccaacgccctctttattctctattacacagcccaaggggagccgttccccaacaacctggacaagctgtgcggccccaatgtgacggacttcccgcccttccacgccaacggcacggagaaggtcaggctggtggagctgtaccgcatcgtggcctaccttggcaccgccctgggcaacatcacccgggaccagaagaccctcaaccccacggcgcacagcctgcacagcaaactcaacgccacggcggacacgctgcggggcctgcttagcaacgtgctgtgccgcctgtgcagcaagtaccacgtggcccacgtggacgtggcctatggcccggacacctcgggcaaggacgtcttccagaagaagaagctggggtgtcagctgctgggaaaatacaagcaggtcatggccgtgttggcgcaggccttctag

SEQ ID NO: 5 - protein, 203 aa, Rabbit
MMKILAAGVVPLLLVLHWKPGAGSPLPINPVNATCNTHHPCPSNLMSQIRSQLAQLNGTANALFILYYTAQGEPFPNNLDKLCGPNVTDFPPFHANGTEKVRLVELYRIVAYLGTALGNITRDQKTLNPTAHSLHSKLNATADTLRGLLSNVLCRLCSKYHVAHVDVAYGPDTSGKDVFQKKKLGCQLLGKYKQVMAVLAQAF

SEQ ID NO: 6 - protein, 202 aa, Homo sapiens
MKVLAAGVVPLLLVLHWKHGAGSPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

SEQ ID NO: 7 - protein, 202 aa, Bovine
MKVLAAGVVPLLLVLHWKHGAGSPLPITPVNATCATRHPCPSNLMNQIRNQLGQLNSSANSLFILYYTAQGEPFPNNLDKLCSPNVTDFPPFHANGTEKARLVELYRIIAYLGASLGNITRDQKVLNPYAHGLHSKLSTTADVLRGLLSNVLCRLCSKYHVSHVDVTYGPDTSGKDVFQKKKLGCQLLGKYKQVIAVLAQAF

SEQ ID NO: 8 - protein, 203 aa, Mouse [sequence omitted]

SEQ ID NO: 9 - protein, 21 aa, Artificial Sequence
  Synthetic construct
LEVLFQGPLGSPNSRVDSSGR

SEQ ID NO: 10 - DNA, 63 bp, Artificial Sequence
  Synthetic construct
ctggaagttctgttccaggggcccctgggatccccgaattcccgggtcgactcgagcggccgc